(19) United States
(12) Patent Application Publication
(10) Pub. No.: US 2015/0299094 A1
(43) Pub. Date: Oct. 22, 2015

(54) PROCESS AND SYSTEM FOR PREPARATION OF ESTERS OF LEVULINIC ACID

(71) Applicant: PRAJ INDUSTRIES LIMITED, Pune, Maharashtra (IN)

(72) Inventors: Jayant Shridhar SAWANT, Pune, Maharashtra (IN); Nitin Sudhakar PATIL, Pune, Maharashtra (IN); Shreekant Uttam SATAV, Pune, Maharashtra (IN); Pallavi Vinitkumar PATHAK, Pune, Maharashtra (IN)

(21) Appl. No.: 14/410,482

(22) PCT Filed: Jul. 10, 2013
(86) PCT No.: PCT/IN2013/000428

§ 371 Date: Dec. 22, 2014

(30) Foreign Application Priority Data
Jul. 19, 2012 (IN) 2074/MUM/2012

(51) Int. Cl. C07C 67/08; B01J 19/24

(52) U.S. Cl. CPC C07C 67/08; B01J 19/24; B01J 2219/00051; B01J 2219/24

(57) ABSTRACT

A process for converting sugars to esters of levulinic acid comprises mixing alcohol and glycerol with a feed stock comprising one or more sugars to create a reaction mixture of alcohol, glycerol and the feedstock is disclosed. The reaction mixture is contacted with an acid catalyst. The contact between the acid catalyst and the reaction mixture is maintained at an effective temperature and pressure for a specific time period so as to cause a desired reaction to result in a product stream. The product stream is separated into components by virtue of the difference in their boiling points, miscibility or specific gravity. Esters of levulinic acid, glycerol, alcohol and additional by-products are recovered from the product stream.

### FIELD OF INVENTION

The invention relates to a process and system for the preparation of levulinic acid esters and more particularly to an elevated temperature conversion of sugar containing feed stocks to alkyl levulinates using acid catalysts.

### BACKGROUND

Ethyl levulinate, a member of alkyl levulinates group, is a keto-ester generally used as octane booster for gasoline and as a fuel extender for diesel. It is also a substrate for a variety of condensation and addition reactions involving the ester and keto groups with alkyl amines to form various polymers which have applications in various industrial processes like coatings, plastic, films, etc as well as a reactant in many chemical preparation processes to create various chemical substances that are used in consumer or pharmaceutical products. Ketals of ethyl levulinate with glycerol find applications as solvents in cleaning formulations and as plasticizers for phthalate replacement.

Conversion of glucose and other hexose sugars to levulinic acid by treating hexose sugar containing streams with mineral acids appeared in the patent literature as early as 1960 (Canadian patent no. CA594974A). This method was adapted later to the direct transformation of biomass using mineral acids at high temperatures. Alternatively, a two step process for the synthesis of levulinate esters was introduced which involved hydrolysis of biomass to levulinic acid followed by its esterification in the second step. Besides, PCT application WO2005/070867 discloses reactive extraction of levulinic acid by means of esterification of C5 to C12 unbranched aliphatic alcohols with yields of levulinate esters as high as 85%. However, most of these methods require the use of corrosive acids and hazardous chemicals during the conversion of sugars to levulinates.

Recently published methods describe the use of heterogeneous catalysts. For example, Peng et al. disclosed a one-pot conversion of glucose to ethyl levulinate over solid catalysts, particularly, sulfated forms of metal oxides, such as zirconia, titania, or zirconia mixed with titania and alumina. [App. Cat. A: Gen 397 (2011) 259-265 and J. Nat. Gas Chem. (2012) 21(2), 138-147]. Saravanamurugan et al. have also reported use of solid acids like sulfonated SBA15 and sulfated zeolites viz. Y, mordenite and ZSM 5 for the same purpose [Chem. Comm (2012), 17, 71-75.]

The major issues with conversion of sugars to ethyl levulinate relate to the selectivity of the conversion, the yields obtained, and the associated high cost of production of ethyl levulinate. Thus, there is still a need for a process to produce levulinic acid and its esters that is economic, energy efficient, environment friendly and yet capable of producing desired products with enhanced purity and significantly increased product yields.

### BRIEF DESCRIPTION OF THE INVENTION

The present invention provides a process for converting sugars to esters of levulinic acid. The present invention also provides a system for producing esters of levulinic acid from one or more sugars or sugar derivates. The present invention further provides a product comprising esters of levulinic acid. Applications for a product comprising esters of levulinic acid are also provided.

A process for converting sugars to esters of levulinic acid, in accordance with one aspect of the invention, comprises mixing an alcohol and glycerol with a feed stock comprising one or more sugars or sugar derivatives like alkyl glycoside, to create a reaction mixture of alcohol, glycerol and the feedstock. The reaction mixture is then contacted with an acid catalyst. The contact between said acid catalyst and the reaction mixture is maintained at an effective temperature and pressure for a specific time period so as to cause a desired reaction to result in a specific product stream. The product stream is separated into components by virtue of the difference in their boiling points, miscibility or specific gravity. Esters of levulinic acid, glycerol, alcohol, and additional by-products are recovered from the product stream.

In accordance with another aspect of the invention, a system for producing esters of levulinic acid from one or more sugars or sugar derivatives like ethyl glucoside, is provided. The system comprises (a) a feed preparation module for mixing a feed stock comprising one or more sugars or its derivatives such as alkyl glycoside with an alcohol and glycerol, creating a reaction mixture of alcohol, glycerol and feed stock and having a desired ratio of alcohol to glycerol and a desired amount of total hexose sugars; (b) a reaction module characterized by a high temperature and pressure vessel for contacting said reaction mixture feed with an acid catalyst at an effective temperature and pressure for a specific time period so as to cause a desired reaction to result in a product stream; and (c) a downstream processing module comprising: (i) a flash tank for separation of said product stream into a vapor phase stream and a liquid-solid phase stream, (ii) a solid-liquid separator for separation of said liquid-solid phase stream to a liquid stream and a solid stream, (iii) an alkali treatment tank to adjust pH of said liquid stream to between about 6 to about 8, (iv) a recovery tank and distillation module for separation and recovery of low boilers from said vapor phase stream, and (v) a primary distillation module and a final distillation module for separation and recovery of components from the liquid stream.

Still another aspect of the invention provides for a product comprising esters of levulinic acid obtained according to a process comprising: (a) mixing alcohol and glycerol with a feed stock comprising one or more sugars or sugar derivatives creating a reaction mixture of alcohol, glycerol and feed stock; (b) contacting said reaction mixture with an acid catalyst; (c) maintaining said solid catalyst contact with said reaction mixture at an effective temperature and pressure for a specific time period so as to cause a desired reaction to result in a product stream; (d) separating said product stream into components by virtue of the difference in their boiling points, miscibility or specific gravity; (e) recovering alcohol and glycerol; (f) recovering esters of levulinic acid and (g) recovering additional by-products.

Yet another aspect of the invention provides for a product comprising alkyl acetate, alkyl formate, diethyl ether or a mixture thereof obtained according to a process comprising: (a) mixing alcohol and glycerol with a feed stock comprising one or more sugars or sugar derivatives, creating a reaction mixture of alcohol, glycerol and feed stock; (b) contacting said reaction mixture with an acid catalyst; (c) maintaining said catalyst contact with said reaction mixture at an effective temperature and pressure for a specific time period so as to cause a desired reaction to result in a product stream; (d) separating said product stream into components by virtue of the difference in their boiling points, miscibility or specific gravity; (e) recovering alcohol and glycerol; (f) recovering esters of levulinic acid and (g) recovering additional by-products.

Another aspect of the invention provides for a product comprising esters of levulinic acid obtained according to a process comprising: (a) mixing alcohol and glycerol with a feed stock comprising one or more sugars creating a reaction mixture of alcohol, glycerol and feed stock; (b) contacting said reaction mixture with an acid catalyst; (c) maintaining said catalyst contact with said reaction mixture at an effective temperature and pressure for a specific time period so as to cause a desired reaction to result in a product stream; (d) separating said product stream into components by virtue of the difference in their boiling points, miscibility or specific gravity; (e) recovering alcohol and glycerol; (f) recovering esters of levulinic acid; and (g) recovering additional by-products, wherein the esters of levulinic acid comprise a starting material for synthesis of polymers, fuel additives, bulk chemicals, solvents, bulk chemicals, detergents, antifreeze agents, pharmaceutical intermediates, herbicides, plasticizers, lubricants, cleaning chemicals or resins.

### DESCRIPTION OF DRAWINGS

These and other features, aspects, and advantages of the present invention will become better understood when the following detailed description is read with reference to the accompanying drawings, wherein:

FIG. 1 depicts a process flow diagram of the production of ethyl levulinate from a feedstock comprising hexose sugars such as glucose or ethyl glucoside. Different elements of the process are identified and directional movement of different streams and components formed during the process are shown to describe the features of one embodiment of the present invention.

FIG. 2 is a schematic diagram of one embodiment of the present invention for producing esters of levulinic acid and additional by-products from feed stock comprising hexose sugars like liquid glucose obtained from corn starch. Different modules as detailed in description are identified and directional movement of different streams and components of the process of the invention are marked. A person skilled in the art may appreciate variations of the system to achieve the conversion of sugars as disclosed herein.

FIG. 3 graphically depicts the effect of different ratios of ethanol to glycerol on the molar yields of ethyl levulinate.

### DETAILED DESCRIPTION OF THE INVENTION

Feed stock comprising one or more hexose sugars or sugar derivatives like ethyl glucoside may be converted in accordance with one aspect of the present invention to form esters of levulinic acid useful for applications such as polymers, bulk chemicals, solvents, fuel additives, detergents, antifreeze agents, pharmaceutical ingredients, herbicides, plasticizers, lubricants, cleaning chemicals or resins. The term hexose sugar is used to generally refer to compounds having the general formula Cₓ(H₂O)_{y}, in which the ratio of hydrogen to oxygen is the same as in water and x is a multiple of 6 and y is equal to x. Hexose sugars may include monosaccharides, polysaccharides and mixtures of monosaccharides and/or polysaccharides. The term “monosaccharide” or “monosaccharides” generally refers to polyhydroxy aldehydes (aldose sugars) or polyhydroxy ketones (ketose sugars) which cannot be hydrolyzed into any simple carbohydrate.

Examples of hexose sugars include disaccharides, oligomers of glucose, polymers of glucose, aldohexoses such as glucose, galactose, mannose, allose, altrose, gulose, iodose, and talose, ketohexoses such as D and L isomers of fructose, psciose, sorbose and tagatose, also glycosides. Sources of hexose sugars may include those derived from sugarcane or sugarbeet or sweet sorghum juice or syrup or molasses, dates, honey, corn, potato, cassava, sago, rice, wheat, barley, sorghum or millet Sources of hexose sugars may also include those derived from lignocellulosic biomass such as agricultural residues of sugarcane, corn, wheat, rice, and palm. Sources of hexose sugars may further include those derived from municipal solid waste.

In one embodiment of the present invention, a process for converting sugar to esters of levulinic acid comprises mixing alcohol and glycerol with a feed stock comprising one of more sugars creating a reaction mixture of alcohol, glycerol and feedstock. The alcohol may comprise methanol, ethanol, n-propanol or n-butanol. The source of glycerol may be synthetic glycerol produced from propylene or bioglycerol obtained as a by-product from industries involved in fat splitting, soap making or production of biodiesel. Crude glycerol may be purified to remove alkali metal impurities and moisture prior to use. The mass ratio of alcohol to glycerol in the reaction mixture is greater than about one. The mass percentage of total hexose sugars in the reaction mixture is in a range from about 0.01 percent to about 25 percent. The mass percentage of water in the reaction mixture may be in a range from about 5 percent to about 10 percent.

In accordance with an embodiment of the present invention, the reaction mixture is contacted with a solid acid catalyst. A contact between the reaction mixture and the solid acid catalyst is maintained at an effective temperature and pressure so as to cause a desired reaction to result in a product stream. The solid acid catalyst may be selected from sulfated zirconia or acid clay. However, catalysts also include, but are not limited to oxides or a mixture of oxides of elements such as aluminum, silicon, zirconium, wherein a surface of catalyst interacts with the reacting species by virtue of accepting electrons [Lewis acidity] or donating protons [Bronsted acidity] to enhance reaction kinetics. Sulfated zirconia catalysts are commercially available and are characterized by their specific pore size, sulfur content and acidity. Sulfated zirconia catalysts of the mesoporous type with good Bronsted acidity and a sulfur content in a range from about 3 mass percent to about 10 mass percent in the catalyst may be used. Numerous types of acid clays such as structured aluminum silicates which are commercially available may be used. Catalytic activity is governed by pore size and surface acidity. Acid clays with a pore size of about 2 nanometers to about 50 nanometers may be used.

In accordance with another embodiment of the present invention, the reaction mixture is contacted with an acid catalyst like methylsulfonic acid [MSA], or sulfuric acid. A contact between the reaction mixture and the acid catalyst is maintained at an effective temperature and pressure so as to cause a desired reaction to result in a product stream.

In one embodiment of the present invention, the contact between the reaction mixture and the acid catalyst is maintained at a temperature in the range from about 140° C. and 220° C. In another embodiment of the present invention, the contact between the reaction mixture and the solid acid catalyst is maintained for a time period ranging from about 20 minutes to about 18 hours.

In one embodiment of the present invention, the reaction results in a product stream comprising esters of levulinic acid, esters of formic acid, esters of acetic acid, dialkyl ethers, levulunic acid, formic acid, acetic acid, and humins Humins are polymeric compounds formed from carbohydrates at elevated temperatures under acidic conditions, having complex structures containing various acyclic and heteroaromatic furanose rings. In another embodiment of the present invention, the product stream is separated into a vapor phase stream and a liquid-solid stream. Esters of formic acid, esters of acetic acid, dialkyl ethers, and a portion of unreacted alcohol are recovered from the vapor phase stream. The liquid-solid stream is separated into a liquid stream and a solid stream. Solid acid catalyst is recovered from the solid stream and may be recycled back into the process. The solid acid catalysts may be activated using well known processes for activation of catalysts before being recycled. In one method of activation of the catalyst, the catalyst may be washed by one or more of solvents like water or ethanol followed by heating at elevated temperatures to remove occluded and adsorbed organic matters.

In one embodiment of the present invention, alkali is added to the liquid stream to adjust pH in a range from about 6 to about 8. The alkalis used may include, but are not limited to aqueous solution of ammonia, metal and alkaline metal hydroxides, such as hydroxides of sodium, potassium, calcium, lithium, and their carbonates and bicarbonates. In another embodiment of the present invention, esters of levulinic acid, unreacted glycerol, another portion of unreacted alcohol and residual by-products are recovered from the liquid stream after pH adjustment. Residual by-products include high boiling components like various acids such as levulinic acid, acetic acid, formic acid, succinic acid and humic acid. In yet another embodiment of the present invention, the unreacted alcohol recovered from the vapor phase stream and the liquid stream may be recycled back to the process. In a further embodiment of the present invention, the unreacted glycerol recovered from the liquid stream may be recycled back to the process.

In a process according to one embodiment of the present invention, esters of levulinic acid are prepared from hexose sugars as detailed below in a system of modules, wherein each module has a specific function leading to conversion of said sugars to the esters of levulinic acid and other additional by-products. In another embodiment of the present invention, the system may be used to recover and recycle of the alcohol and glycerol used in the process. The ethyl levulinate so afforded can be hydrolyzed to obtain levulinic acid.

In one embodiment of the present invention, efficient conversion of hexose sugars to ethyl levulinate and additional by-products like ethyl formate, ethyl acetate, diethyl ether and levulinic acid are obtained using a system comprising three modules namely: 1] feed preparation module; 2] reaction module; and 3] downstream processing module. Each module has one or more elements for performing specific or optional functions as required for achieving selective production of ethyl levulinate at higher amounts. A person skilled in the art may appreciate different variations and/or combinations of these elements that may be used to perform the objects of the invention disclosed herein.

### Feed Preparation Module

Feed preparation module is used for mixing a feed stock comprising one or more hexose sugars or sugar derivatives with an alcohol and glycerol and adjusting moisture content to a desired level if necessary. This is followed by mixing leading to the formation of a reaction mixture of feed stock, alcohol and glycerol in a desired ratio of alcohol to glycerol and a desired amount of hexose sugar in the mixture leading to a desired composition of the reaction mixture. The reaction mixture obtained in the feed preparation module is fed to the next module called reaction module.

In one embodiment of the present invention, a feed stock comprising glucose like liquid glucose obtained from corn starch is mixed with ethanol and glycerol in the feed preparation module leading to the creation of a reaction mixture for the preparation of ethyl levulinate at higher amounts and additional by-products like ethyl formate, ethyl acetate and diethyl ether. Other feed stocks like C6 fraction of corn cob hydrolyzate [cellulosic part] and ethyl glucoside obtained from glucose may be used.

In another embodiment of the present invention, a feed stock comprising glucose is mixed with methanol and glycerol in the feed preparation module leading to the creation of a reaction mixture for the preparation of methyl levulinate at higher amounts and additional by-products like methyl formate, methyl acetate and dimethyl ether.

In yet another embodiment of the present invention, a feed stock containing glucose is mixed with n-butanol and glycerol in the feed preparation module leading to the creation of a reaction mixture for the preparation of n-butyl levulinate at higher amounts and additional by-products like n-butyl formate, n-butyl acetate and n-dibutyl ether.

### Reaction Module

The second module of the system is a reaction module characterized by one or more high temperature and pressure vessels, capable of holding the reaction mixture, solid acid catalyst and a product stream and allowing a desired reaction to occur at a condition of high pressures up to 60 bars and high temperatures up to 300° C. These vessels may be one or more of stirred tanks, plug-flow reactors, accelerated plug-flow reactors, recirculation reactors or fluidized-bed reactors. These vessels may operate in batch mode, fed-batch mode or continuous mode in parallel or in series or a combination thereof. The vessels also contain an acid catalyst for efficient contact of the acid catalyst with the reaction mixture for efficient conversion of hexose sugars to esters of levulinic acid and other products as described herein. The reaction module has means for controlling temperature and pressure at a desired level during the reaction for extended time periods. In one embodiment of the present invention, the reaction mixture and the acid catalyst are fed into the reaction module and contact between the catalyst and the reaction mixture is maintained at an effective temperature and pressure for a specific retention time period in the vessels to achieve a desired reaction resulting in formation a product stream, which is fed to the next module called downstream processing module.

In one embodiment of the present invention, the vessel is an autoclave vessel wherein the reaction mixture is subjected to an elevated temperature and pressure for a desired time period in the presence of an acid catalyst held in place in the autoclave such that it optimally contacts the reaction mixtures leading to efficient production of desired products.

In another embodiment of the present invention, the vessel is a batch-type stirred tank reactor wherein a mixture of fructose, ethanol and glycerol are subjected to an elevated temperature and pressure for a desired time period in the presence of an acid catalyst such that it optimally contacts the reaction mixture leading to efficient production of ethyl levulinate and other by-products.

In yet another embodiment of the present invention, the vessel is a plug-flow reactor wherein a mixture of soluble starch, ethanol and glycerol are subjected to an elevated temperature and pressure for a desired time period in the presence of a solid acid catalyst such that it optimally contacts the reaction mixture leading to efficient production of ethyl levulinate and other by-products.

In yet another embodiment of the present invention, the vessel is a recirculation reactor wherein a mixture of liquid glucose, ethanol and glycerol are subjected to an elevated temperature and pressure for a desired time period in the presence of a solid acid catalyst such that it optimally contacts the reaction mixture leading to efficient production of ethyl levulinate and other by-products.

### Downstream Processing Module

The third module of the system is called downstream processing module and is characterized by having several elements that efficiently separate the multiple components of the product stream coming from the reaction module that are present in vapor, liquid and solid phases. As first act, the product stream from the reaction module is subjected to a pressure in a flash tank which is lower than the pressure in the vessel, which forms the first element of the downstream processing module. In the flash tank, the vapor phase stream of the product stream is separated, and the vapor phase stream is condensed to a condensed vapor phase stream in a condenser. The condensed vapor phase stream is subjected to a low boiler distillation to separate the additional by-products like akyl formate, alkyl acetate and dialkyl ether from a secondary alcohol component. The secondary alcohol component is recovered, rectified and recycled back to the system. The vapor phase condensation and distillation of the condensed vapor phase stream forms the second element in this module. A liquid-solid stream which is part of the product stream in the flash tank is subjected to a solid-liquid separation leading to separation of solid acid catalyst from a liquid stream. In another embodiment of the invention, the liquid-solid stream is subjected to a solid-liquid separation technique including, but not limited to, filtration or centrifugation or decantation. The solid catalyst may be optionally activated and recycled.

The third element of the module is the separation of products from a liquid stream obtained from the product stream after separation of vapor phase components and solid materials. The liquid stream obtained from the solid-liquid separator may be subjected to an alkali treatment process in an alkali treatment tank, wherein pH of the liquid stream is adjusted between about 6 to about 8 by addition of a desired alkali to form a lean alkyl levulinate stream. The lean alkyl levulinate stream is subjected to a primary distillation leading to separation of the lean alkyl levulinate stream into a primary alcohol component and a crude alkyl levulinate stream. The primary distillation may be done in a single primary distillation module or multiple primary distillation modules. The crude alkyl levulinate stream is further subject to a final distillation leading to separation of the crude alkyl levulinate stream into alkyl levulinate and a glycerol containing stream. The final distillation may be done in a single final distillation module or multiple final distillation modules. The glycerol stream is further processed to recover residual by-products and glycerol that may be recycled back to the system. The primary alcohol component is also recovered and may be recycled back to the system after rectification. The other elements of the downstream processing module include suitable unit operations for the processing, separation and recovery of products, and means for control and monitoring these operations. A person skilled in the art may appreciate various aspects of said system and any combinations of the known unit operations to affect the objects of the invention disclosed herein.

The ethyl levulinate content at the various stages in the process was analyzed in test samples. Ethyl levulinate was analyzed by gas chromatography with flame ionization detector. Alltech ATTM-wax column of size 60 m×0.53 mm ID and film thickness of 1 μm was used. Helium was used as carrier gas with flow rate of 3 mL/min Injector and detector temperatures were kept at 240° C. and 260° C. respectively. Temperature gradient program was used with total run time of 24 minute. Retention time of ethyl levulinate was observed at about 10.63 minute. Sample injection volume was 1 μL. Dimethyl Formamide was used as a diluent for the sample preparation. Estimation of ethyl levulinate content in the test samples was done using calibration graph generated using at least five known concentrations of standard compound.

Glucose content was analyzed by liquid chromatography with refractive index detector. BioRad Aminex 87 H⁺ column of size 300×7.8 mm was used with 0.005 M H₂SO₄ as mobile phase with flow rate of 0.6 mL/min Column oven temperature was kept at 55° C. and injection volume at 20 μL. Deionised water was used as a diluent for the sample preparation. Retention time of glucose was observed at 8.9 minute. Estimation of glucose content in the test samples was done using calibration graph generated using five known concentration of standard compound

Representative features of the invention are illustrated in the DRAWINGS.

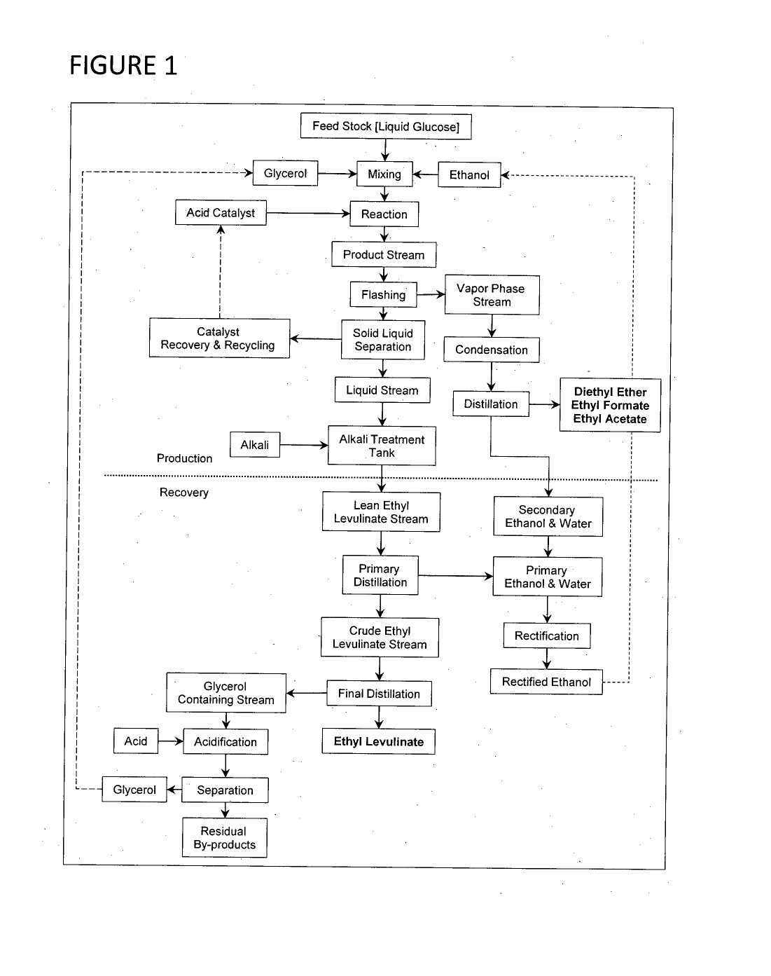

FIG. 1 depicts a process flow for the conversion of a feed stock comprising one or more sugars to ethyl levulinate and additional by-products in accordance with one aspect of the present invention. In the first act a feed stock comprising one or more sugars is mixed with glycerol and ethanol to create a reaction mixture. The reaction mixture is subjected to an elevated temperature and pressure in the presence of an acid catalyst leading to formation of a product stream. The product stream is subjected to flashing to separate a vapor phase stream from a liquid-solid stream. The vapor phase stream is subjected to condensation and distillation leading to separation of additional by-products like diethyl ether, ethyl formate and ethyl acetate from a secondary ethanol component. The secondary ethanol component may be rectified and recycled. The liquid-solid stream is separated to obtain a liquid stream and a solid stream. The solid catalyst is separated from the solid stream and may be recycled. The liquid stream is treated with an alkali to adjust pH in a range between about 6 to about 8 to obtain a lean ethyl levulinate stream. This stream is subjected to a primary distillation to obtain crude ethyl levulinate stream and separate a primary ethanol component. The primary ethanol component may be rectified and recycled. The crude ethyl levulinate stream is subjected to final distillation to separate ethyl levulinate from a glycerol containing stream. The glycerol containing stream is acidified to separate residual by-products from glycerol. The glycerol obtained may be recycled back to the system.

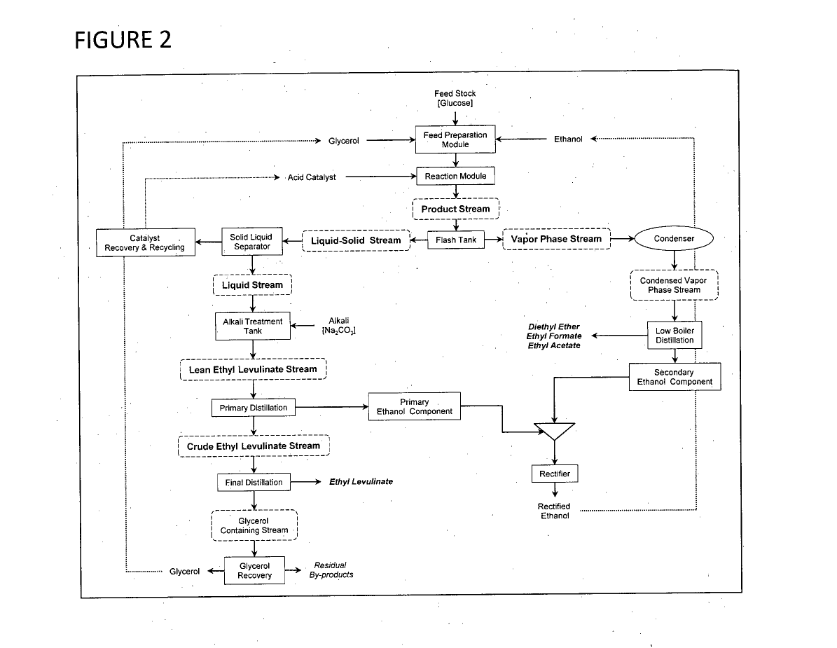

FIG. 2 depicts system elements used in an embodiment of the present invention for the preparation of ethyl levulinate and additional by-products from a feed stock containing glucose as hexose sugar. In the first module termed “Feed Preparation Module” in FIG. 2, feed stock, ethanol and glycerol are mixed to create a reaction mixture of desired properties. This reaction mixture is subject to a high temperature and pressure condition in the second module termed “Reaction Module” in FIG. 2. The temperature in the Reaction Module is maintained between about 140° C. to about 220° C. leading to formation of a product stream, which is directed to the first element of third module called flash tank at a pressure lower than the reaction module. In the flash tank, a vapor phase stream is separated, further condensed and subjected to a low boiler distillation leading to separation of ethyl formate, ethyl acetate and diethyl ether as additional products. The ethanol from this stream is collected as secondary alcohol component and is optionally recycled after rectification. From the flash tank, a liquid-solid stream is separated in solid-liquid separator, removing a solid acid catalyst and leading to formation of a liquid stream. The liquid stream is subjected to a pH adjustment in an alkali treatment tank leading to formation of a lean ethyl levulinate stream. The lean ethyl levulinate stream is subjected to a primary distillation leading to separation of a primary ethanol component from a crude ethyl levulinate stream. The primary ethanol component is optionally subjected to rectification and recycled back to the system. This crude levulinate stream is further subjected to a final distillation leading to separation of ethyl levulinate from a glycerol containing stream. Glycerol containing stream is further processed to recover glycerol and separate residual by-products like acetic acid, formic acid and levulinic acid. Glycerol recovered is optionally recycled back to the system.

### EXAMPLES

Examples provided below give wider utility of the invention without any limitations as to the variations that may be appreciated by a person skilled in the art. A non-limiting summary of various experimental results is given in the examples and tables, which demonstrate the advantageous and novel aspects of the process of using glycerol at elevated temperatures in a pressure vessel for efficient conversion of hexose sugars to esters of levulinic acid.

The characteristics of the suitable starting material in the above described reaction process for producing the desired ethyl levulinate are summarized in Table 1.

TABLE 1
Physical and chemical specifications of liquid glucose an d C6 cake

| | Specification of | Specification |
|---|---|---|
| | industrial | of C6 cake |
| | grade liquid | [cellulose |
| | glucose from | fraction] from |
| Parameter | corn starch | corn cob |
| Moisture percent by weight | 13 to 18 | 65 to 75 |
| Total solids at 20° C. percent by mass | 82 to 87 | 25 to 35 |
| Sulfated ash percent by mass (Max) | 0.50 | — |
| pH of 50% solution at 25° C. | 4.50 to 5.50 | 1.0 to 2.0 |
| Dextrose equivalent [% by dry weight] | 38 to 48 | 65 to 75 |

### Example 1

48 gram of glucose in the form of commercial liquid glucose (85.5% solids, dextrose equivalence 41.75% w/w) was mixed with 210 gram ethanol and 90 gram of glycerol. This mixture was transferred to a stirred tank reactor; 6 gram of commercially available sulfated zirconia [7% sulfur content] was added as a solid acid catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 50.95%. In examples herein molar yield is defined as percent ratio of product moles obtained to moles of reactant. Molar yield is used through out, is calculated as:

Percent Molar Yield=(Weight of Ethyl Levulinate×Molecular Weight of Glucose×100)/(Weight of Glucose Reacted×Molacular Weight of Ethyl Elevulinate). Molar yeild at theoretical maxium for conversion of glucose to ethyl levulinate is 1.

### Example 2

80 gram of fructose (extra pure grade obtained from Merck) was mixed with 278 gram of ethanol and 42 gram of glycerol. This mixture was transferred to a stirred tank reactor; 6 gram of commercially available sulfonated zirconia [10% sulfur content] was added as a catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 47.46%

### Example 3

27 gram of glucose in the form of synthetic dextrose powder (94% solids, dextrose equivalence 96% w/w) was mixed with 210 gram of ethanol and 90 gram of glycerol. This mixture was transferred to a stirred tank reactor, 3 gram of sulfated zirconia catalyst [7% sulfur content; activated at 350° C. for two hours] was added to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 42.13%.

### Example 4

80 gram of glucose in the form of commercial liquid glucose (85.5% solids, dextrose equivalence 41.75% w/w) was mixed with 340 gram of ethanol and 60 gram of glycerol. This mixture was transferred to a stirred tank reactor; 10 gram of montmorilinite clay KSF (obtained from Sigma Aldrich) was added as a catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 50.07%.

### Example 5

37 gram of glucose in the form of dextrose powder (94% solids, dextrose equivalence 96% w/w) was mixed with 112 gram of ethanol and 48 gram of glycerol. This mixture was transferred to a stirred tank reactor; 4 gram of commercially available sulfated zirconia [10% sulfur content] was added as a catalyst to the reaction mass in the reactor. The reactor was closed and then heated under stirred condition to reach a temperature of 200° C. The reactor was maintained at this temperature for four hours. After four hours the reactor was cooled and the reaction mass was analyzed for ethyl levulinate formation by gas chromatography. Molar yield of ethyl levulinate was found to be 42.18%.

### Example 6

36 gram of glucose in the form of dextrose powder (94% solids, dextrose equivalence 96% w/w) was mixed with 140 gram of ethanol and 60 gram of glycerol. This mixture was transferred to a stirred tank reactor; 4 gram of commercially available sulfated zirconia [10% sulfur content] was added as a catalyst to the reaction mass in the reactor. The reactor was closed and then heated under stirred condition to reach a temperature of 180° C. The reactor was maintained at this temperature for two hours. After two hours the reactor was further heated to reach a temperature of 220° C. This temperature was maintained for another two hours. The reactor was then cooled and the reaction mass was analyzed for ethyl levulinate formation by gas chromatography. Molar yield of ethyl levulinate was found to be 39.59%.

### Example 7

1041 gram of glucose in the form of commercial liquid glucose (85.5% solids, dextrose equivalence 41.75% w/w) was mixed with 4423 gram of ethanol and 780 gram of glycerol. This mixture was transferred to a stirred tank reactor; 130 gram of commercially available sulfated zirconia [7% sulfur content] was added as a catalyst to the reaction mass in the reactor. The reactor was closed and then heated under stirred condition to reach a temperature of 160° C. The reactor was maintained at this temperature for one hour. After one hour the reactor was further heated to reach a temperature of 210° C. This temperature was maintained for another three hours. The reactor was then cooled and filtered to separate the catalyst. The catalyst was further subjected to recycle studies as described herein. The filtrate was analyzed for ethyl levulinate formation by gas chromatography. Molar yield of ethyl levulinate was found to be 47.18%.

### Example 8

45 gram of glucose in the form of dextrose powder (94% solids, dextrose equivalence 96% w/w) was mixed with 315 gram of ethanol and 135 gram of glycerol and 50 gram of water. This mixture was transferred to a stirred tank reactor; 5 gram of commercially available sulfated zirconia [10% sulfur content] was added as a catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 38.13%.

### Example 9

27 gram of glucose in the form of dextrose powder (94% solids, dextrose equivalence 96% w/w) was mixed with 55 gram of methanol and 245 gram of glycerol. This mixture was transferred to a stirred tank reactor; 3 gram of commercially available sulfated zirconia [10% sulfur content] was added as a catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for methyl levulinate formation by gas chromatography. Molar yield of methyl levulinate was found to be 15%.

### Example 10

27 gram of glucose in the form of synthetic dextrose powder (94% solids, dextrose equivalence 96% w/w) was mixed with 79 gram of ethanol and 221 gram of glycerol. This mixture was transferred to a stirred tank reactor; 3 gram of sulfonated zirconia catalyst [7% sulfur content; activated at 350° C. for two hours] was added to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 14.52%.

### Example 11

Hexose containing feedstock was mixed with an alcohol and glycerol. The mixture was contacted with a catalyst in a stirred tank rector at a temperature of 210° C. for four hours. Catalyst used was sulfated zirconia [10% sulfur content]. The amount of catalyst used was 12.5% w/w of the feed. After four hours the reaction mass was analyzed by gas chromatography for formation of alkyl esters of levulinic acid. A similar control batch was conducted for each alcohol wherein all conditions were kept same except for addition of glycerol. The results of these experiments are shown in Table A.

TABLE A
effect of glycerol as a co-solvent along with various alcohols.

| | | Composition of | | | | Molar yield of alkyl |
|---|---|---|---|---|---|---|
| | | feed mixture (% w/w) | | | | levulinate % of |
| Ex. | Type of | Sug- | Alco- | Glyc- | Wa- | theoretical |
| No. | alcohol | ar | hol | erol | ter | maximum |
| 1. | Methanol | 15.7 | 56 | 24 | 4 | 44.5 |
| 2. | Methanol | 16 | 80 | 0 | 4 | 31.7 |
| 3. | Ethanol | 8.2 | 63.6 | 27.2 | 1 | 42.13 |
| 4. | Ethanol | 8.2 | 90.1 | 0 | 1 | 20.8 |
| 5. | n-butanol | 16 | 56 | 24 | 4 | 14.4 |
| 6. | n-butanol | 16 | 80 | 0 | 4 | 3.8 |

### Example 12

Experiments were conducted to demonstrate the conversion of different feed stock comprising hexose sugars to esters of levulinic acid. The procedure used was similar to that in Example 12, wherein for each experiment a different feedstock was mixed with ethanol and glycerol. The mixture was contacted with a catalyst in a stirred tank reactor at a temperature 210° C. for a certain period of time after which the reaction mass was analyzed by gas chromatography for the formation of ethyl ester of levulinic acid. Catalyst used for these experiments was sulfated zirconia (10% SO₃) and the amount of catalyst used was about 12.5% w/w of the feed. The results of these experiments are shown in Table B.

TABLE B
Effect of glycerol as a co-solvent with ethanol for different
hexose feed stocks during formation of ethyl levulinate.

| | | | | | | | Molar yield |
|---|---|---|---|---|---|---|---|
| | | Composition of | | | | | of ethyl |
| | | reaction mixture (% w/w) | | | | Reaction | levulinate % of |
| Ex. | Feed- | Sug- | Etha- | Glyc- | Wa- | time | theoretical |
| no. | stock | ar | nol | erol | ter | (hours) | maximum |
| 1 | Dextrose | 15.2 | 58.3 | 25.0 | 3.9 | 4 | 37.7 |
| | powder | | | | | | |
| 2 | Fructose | 19.6 | 68.1 | 10.3 | 2 | 3 | 47.5 |
| | powder | | | | | | |
| 3 | Liquid | 13.5 | 58.9 | 25.2 | 2.5 | 4 | 51.0 |
| | glucose | | | | | | |
| 4 | Soluble | 8.2 | 63.6 | 27.3 | 1 | 4 | 35.1 |
| | Starch | | | | | | |

### Example 13

Experiments were conducted to illustrate the applicability of invention to various catalysts. The procedure used was similar to that used in Example 12 wherein for each experiment, liquid glucose from a corn starch was mixed with ethanol and glycerol, the mixture was then contacted with a catalyst in a stirred tank rector at a temperature 210° C. for four hours. After four hours the reaction mass was analyzed by gas chromatography for formation of ethyl ester of levulinic acid. Catalyst used in each of the experiments and the yield of ethyl levulinate is shown in Table C. The amount of catalyst used was 12.5% w/w of the feed.

TABLE C
Performance of various catalysts in glycerol/ethanol system.

| | | | | | | Molar yield |
|---|---|---|---|---|---|---|
| | | Composition of | | | | of ethyl |
| | | feed mixture (% w/w) | | | | levulinate % of |
| Ex. | Name of | Sug- | Etha- | Glyc- | Wa- | theoretical |
| no. | catalyst | ar | nol | erol | ter | maximum |
| 1. | Sulfated zirconia | 20 | 64 | 12 | 4 | 40.16 |
| | (10% SO₃) | | | | | |
| 2. | Sulfated zirconia | 20 | 64 | 12 | 4 | 50.91 |
| | (7% SO₃) | | | | | |
| 3. | Montmorilinite KSF | 16.2 | 68.7 | 12.1 | 3 | 50.1 |
| 4. | MSA | 15.9 | 67.7 | 12 | 4.2 | 45 |
| 5. | Sulfuric acid | 21 | 62 | 12 | 2 | 26 |

### Example 14

Sulfated zirconia (7% SO₃) recovered from Experiment #2 of Example 13 by means of filtration was calcined at 350° C. for two hours and was reused in another experiment which was conducted using a procedure similar to that of Experiment #2 of Example 15. The molar yield of ethyl levulinate was observed to be 28.8%.

### Example 15

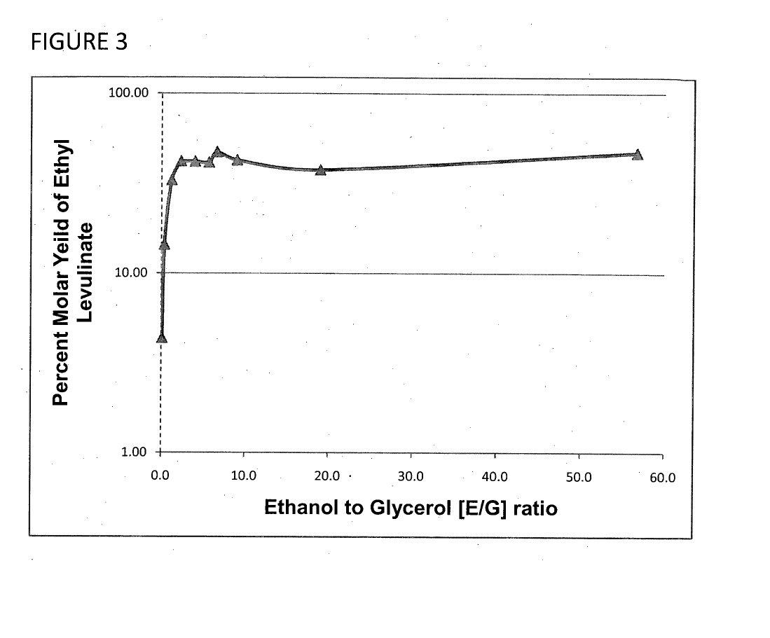

Feed stock comprising hexose sugars was mixed with ethanol and glycerol such that the mass concentration of hexose sugar in the resultant mixture was about 10 to 12.5%. The mixture was contacted with a catalyst in a stirred tank rector at a temperature of about 210° C. for four hours. Catalyst used was sulfated zirconia. The amount of catalyst used was about 12.5% w/w of the feed. After four hours the reaction mass was analyzed by gas chromatography for the formation of ethyl levulinate. Various experiments were carried out wherein for each experiment a different ratio of ethanol to glycerol was used. The results of these batches are shown in Table D. A graphical representation of the results is shown in FIG. 3.

TABLE D
glucose to ethyl levulinate conversions
at various ratios of ethanol to glycerol.

| | Ethanol to glycerol mass ratio | Molar yield of ethyl levulinate |
|---|---|---|
| Ex. no. | (E/G ratio) | % of theoretical maximum |
| 1 | 56.7 | 47.18 |
| 2 | 19.0 | 37.71 |
| 3 | 9.0 | 42.75 |
| 4 | 6.6 | 47.46 |
| 5 | 5.7 | 41.58 |
| 6 | 4.0 | 41.99 |
| 7 | 2.3 | 42.13 |
| 8 | 2.3 | 42.13 |
| 9 | 1.2 | 33.13 |
| 10 | 0.4 | 14.52 |
| 11 | 0.1 | 4.37 |
| Control | No glycerol | 20.0 |

### Example 16

80 gram of glucose in the form of commercial liquid glucose (85.5% solids, dextrose equivalence 41.75% w/w) was mixed with 340 gram ethanol and 60 gram of glycerol. This mixture was transferred to a stirred tank reactor; 1 gram of commercially available methylsulfonic acid [MSA] was added as an acid catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 45%.

### Example 17

69 gram of ethyl glucoside was mixed with 231 gram ethanol and 60 gram of glycerol. This mixture was transferred to a stirred tank reactor; 2.4 gram of commercially available methylsulfonic acid [MSA] was added as an acid catalyst to the reaction mass in the reactor. The reactor was closed and then heated under stirred condition at 170° C. for one hour. Then reactor was maintained at 140° C. for three hours. After four hours the reactor was cooled and the reaction mass was analyzed for ethyl levulinate formation by gas chromatography. Molar yield of ethyl levulinate was found to be 29%.

### Example 18

87 gram of ethyl glucoside was mixed with 266 gram ethanol and 49 gram of glycerol. This mixture was transferred to a stirred tank reactor; 0.75 gram of commercially available sulfuric acid was added as an acid catalyst to the reaction mass in the reactor. The reactor was closed and then heated under stirred condition to reach a temperature of 170° C. The reactor was maintained at this temperature for four hours. After four hours the reactor was cooled and the reaction mass was analyzed for ethyl levulinate formation by gas chromatography. Molar yield of ethyl levulinate was found to be 26%.

### Example 19

30 gram of C6 cake from corn cob was mixed with 308 gram ethanol and 61 gram of glycerol. This mixture was transferred to a stirred tank reactor; 4 gram of MSA was added as an acid catalyst to the reaction mass in the reactor. The reactor was closed and then heated under stirred condition to reach a temperature of 210° C. The reactor was maintained at this temperature for four hours. After four hours the reactor was cooled and the reaction mass was analyzed for ethyl levulinate formation by gas chromatography. Molar yield of ethyl levulinate was found to be 28%.

While the invention has been particularly shown and described with reference to embodiments listed in examples, it will be appreciated that several of the above disclosed and other features and functions, or alternatives thereof, may be desirably combined into many other different systems or applications. Also that various presently unforeseen and unanticipated alternatives, modifications, variations, or improvements therein may be subsequently made by those skilled in the art which are also intended to be encompassed by the following claims. Although the invention has been described with reference to specific preferred embodiments, it is not intended to be limited thereto, rather those having ordinary skill in the art will recognize that variations and modifications may be made therein which are within the spirit of the invention and within the scope of the claims.

1. A process for converting sugars to esters of levulinic acid comprising:
(a) mixing alcohol and glycerol with a feed stock comprising one or more sugars sufficient to create a reaction mixture of alcohol, glycerol and feed stock;
(b) contacting said reaction mixture with an acid catalyst;
(c) maintaining said catalyst contact with said reaction mixture at an effective temperature and pressure for a specific time period so as to cause a desired reaction to result in a product stream;
(d) separating said product stream into components by virtue of the difference in one or more of their boiling points, miscibility and specific gravity;
(e) recovering alcohol and glycerol;
(f) recovering esters of levulinic acid; and
(g) recovering additional by-products.

2. The process of claim 1, wherein said reaction mixture comprises alcohol to glycerol in a mass ratio of greater than about one, and further comprises a mass percentage of total hexose sugars from about 0.01% to about 25%.

3. The process of claim 1, wherein said one or more sugars comprise disaccharides, oligomers or polymers comprising one or more hexose sugars.

4. The process of claim 1, wherein said sugar comprises ethyl glucoside.

5. The process of claim 1, wherein said one or more sugars comprises one or more of aldohexose or ketohexose sugars.

6. The process of claim 1, wherein said one or more sugars comprises one or more of glucose, dextrose, sucrose or fructose.

7. The process of claim 1, wherein said alcohol comprises 1 to 4 carbon atoms.

8. The process of claim 1, wherein said alcohol is ethanol.

9. The process of claim 1, wherein said effective temperature ranges from about 140° C. to about 220° C.

10. The process of claim 1, wherein said specific time period ranges from about 20 minutes to about 18 hours.

11.-12. (canceled)

13. The process of claim 1, wherein a pH of said product stream is adjusted to between about 6 and about 8.

14.-15. (canceled)

16. The process of claim 1, wherein a vapor phase stream, of the product stream, is condensed into a condensed vapor stream and said condensed vapor stream is subjected to a low boiler distillation to separate a secondary alcohol component, alkyl formate, alkyl acetate and dialkyl ether.

17. (canceled)

18. The process of claim 1, wherein said additional by-products comprise alkyl formate, alkyl acetate or dialkyl ethers.

19. (canceled)

20. The process of claim 1, wherein said catalyst is selected from sulfated zirconia, acid clays, methylsulfonic acid or sulfuric acid.

21. A system for producing esters of levulinic acid from a feed stock with one or more sugars or sugar derivatives, comprising:
(h) a feed preparation module for mixing said feed stock with an alcohol and glycerol, sufficient to create a reaction mixture of alcohol, glycerol and feed stock and having a desired ratio of alcohol to glycerol and a desired amount of total hexose sugars in said reaction mixture;
(i) a reaction module characterized by a high temperature and pressure vessel for contacting said reaction mixture with an acid catalyst at an effective temperature and pressure for a specific time period so as to cause a desired reaction to result in a product stream; and
(j) a downstream processing module comprising:
(i) a flash tank for separation of said product stream into a vapor phase stream and a liquid-solid phase stream,
(ii) a solid-liquid separator for separation of said liquid-solid phase stream into a liquid stream and a solid stream,
(iii) an alkali treatment tank to adjust pH of said liquid stream to between about 6 to about 8,
(iv) a recovery tank and distillation module for separation and recovery of low boilers from said vapor phase stream, and
(v) a primary distillation module and a final distillation module for separation and recovery of components from said liquid stream.

22. (canceled)

23. The system of claim 21, wherein said reaction mixture comprises alcohol to glycerol in a mass ratio of greater than about one and further comprises a mass percentage of total hexose sugar from about 0.01% to about 25%.

24.-29. (canceled)

30. The system of claim 21, wherein said effective temperature ranges from about 140° C. to about 220° C.

31. The system of claim 21, wherein said specific time period ranges from about 20 minutes to about 18 hours.

32.-34. (canceled)

35. The system of claim 21, wherein said liquid stream is subjected to a primary distillation and a final distillation to separate alkyl levulinate, a primary alcohol component and a glycerol containing stream.

36.-38. (canceled)

39. The system of claim 21, wherein said reaction module comprises one or more vessels and the vessels are one or more of stirred tank, plug-flow, accelerated plug-flow, recirculation or fluidized-bed reactors.

40.-43. (canceled)
